(19) United States
(12) Patent Application Publication
(10) Pub. No.: US 2022/0047169 A1
(43) Pub. Date: Feb. 17, 2022

(54) FLUORO-ACOUSTIC MULTIPIPETTE ELECTRODE AND METHODS OF USE THEREFOR

(71) Applicants: Barbara S. SMITH, Tempe, AZ (US); Christopher MIRANDA, Mesa, AZ (US)

(72) Inventors: Barbara S. SMITH, Tempe, AZ (US); Christopher MIRANDA, Mesa, AZ (US)

(73) Assignee: Arizona Board of Regents on behalf of Arizona State University, Scottsdale, AZ (US)

(21) Appl. No.: 17/401,640

(22) Filed: Aug. 13, 2021

Related U.S. Application Data
(60) Provisional application No. 63/065,060, filed on Aug. 13, 2020.

(51) Int. Cl. A61B 5/00

(52) U.S. Cl. CPC A61B 5/0095; A61B 2576/026; A61B 5/0042

(57) ABSTRACT

A system for automated navigation to a target neuron is disclosed. The system comprises a recording electrode including a pipette with a hollow glass tip and a headstage for detecting electrical resistance measurements at the glass tip. The system further comprises an actuator, a light source configured to emit light from the glass tip, an ultrasound transducer for detecting photoacoustic signals in response to the light, a light sensor for detecting optical signals in response to the light, and a processor. The processor iteratively receives the photoacoustic and optical feedback and moves the glass tip via the actuator based on a calculated distance of the target neuron. When the distance at or below a predetermined threshold, the processor maintains the position of the hollow glass tip with respect to the target neuron. Upon successful navigation, the recording electrode may be used to perform single-unit neural recording of the target neuron.

### CROSS-REFERENCE TO RELATED APPLICATIONS

This application claims the benefit of priority to U.S. Provisional Application No. 63/065,060 entitled “Fluoro-Acoustic Multipipette Electrode and Methods of Use Therefor,” filed Aug. 13, 2020, which is incorporated herein by reference in its entirety.

### GOVERNMENT INTERESTS

This invention was made with government support under Award No. 1944846 awarded by the National Science Foundation. The government has certain rights in the invention.

### TECHNICAL FIELD

The present disclosure relates generally to methods, systems, and apparatuses related to automated feedback systems for neuron hunting. The disclosed techniques may be applied to, for example, patch clamp electrodes for localizing to target neurons in animals or tissue samples. More particularly, the present disclosure relates to methods, systems, and apparatuses for fluoro-acoustic feedback and navigation of patch clamp electrodes.

### BACKGROUND

For many years, whole-cell patch clamp electrophysiology has served as a gold standard technique for studying the biophysical behavior of neurons in vivo. Whole-cell patch clamp electrophysiology utilizes glass micropipettes to establish molecular and electrical communication with the interior of neurons in intact tissue. This technique facilitates assessment of individual neurons with high temporal resolution in order to analyze small fluctuations in voltage below the membrane's threshold potential, which may be characteristic of specific conditions and behaviors of neural tissue. Accordingly, patch clamp electrophysiology is a crucial tool in further understanding various mechanisms related to neuronal communication and neurological conditions (e.g., addiction) or diseases.

However, establishing whole-cell patch clamping in vivo and in vitro may be very difficult in many cases. In order to localize to a neuron within tissue, patch clamp systems rely on an incremental approach of collecting resistance measurements at a tip of the recording electrode to indicate a relative location of a target neuron and adjusting the position of the recording electrode towards the target neuron. Through a repetitive process of measuring and repositioning, the recording electrode may be localized to the target neuron within the tissue. Performing this process manually is extremely laborious and has a low success rate even with the high level of skill, knowledge and careful decision making required to perform patch clamping. As such, it is difficult to perform frequent experiments to elucidate neuron behavior. Further, simultaneous patch clamping of multiple neurons (e.g., to record several neurons along a signal pathway) is practically infeasible due to the low success rate of the neuron targeting.

Currently available solutions include computer systems that perform automated patch clamping (i.e., “autopatching”) through incremental, computer-controlled movements of the recording electrode based on the resistance feedback. However, resistance measurements may only provide appropriate feedback over a limited range (e.g., several microns) and thus may still experience frequent failure. Further, while image-guided navigation systems have been developed, such systems rely on external equipment (e.g., DIC, confocal microscopy, and/or two-photon microscopy) and are not capable of obtaining targeted recordings at tissue depths of 1 mm or greater due to optical scattering in the tissue.

As such, it would be advantageous to have an automated patch clamping tool that is capable of receiving feedback over a greater range and localizing to target neurons at greater depths beyond the currently available systems.

### SUMMARY

This summary is provided to comply with 37 C.F.R. § 1.73. It is submitted with the understanding that it will not be used to interpret or limit the scope or meaning of the present disclosure.

A system for navigating to a target neuron is provided. The system comprises a recording electrode including: a pipette having a hollow glass tip, and a headstage configured to detect electrical resistance measurements at the hollow glass tip; an actuator configured to move the hollow glass tip in one or more degrees of freedom; at least one light source coupled to the recording electrode and configured to emit light from the hollow glass tip; an ultrasound transducer configured to detect one or more photoacoustic signals in response to the light; a light sensor configured to detect one or more optical signals in response to the light; a processor; and a non-transitory, computer-readable medium storing instructions that, when executed, cause the processor to: perform one or more first iterations, each first iteration comprising: receiving a first set of signals associated with the one or more photoacoustic signals from the ultrasound transducer, receiving a second set of signals associated with the one or more optical signals from the light sensor, calculating, based on at least one of the first set of signals and the second set of signals, a distance of the hollow glass tip with respect to the target neuron, responsive to a determination that the distance is greater than a predetermined threshold, moving the hollow glass tip by a first increment in the one or more degrees of freedom via the actuator based on the distance, and responsive to a determination that the distance is less than or equal to the predetermined threshold, maintaining the position of the hollow glass tip with respect to the target neuron via the actuator.

According to some embodiments, the system further comprises a suction source communicating with the hollow glass tip, wherein the instructions, when executed, further cause the processor to activate the suction source, thereby forming a gigaohm seal between the hollow glass tip and the target neuron. According to additional embodiments, the instructions, when executed, further cause the processor to control the suction source and the actuator to form one of a whole-cell patch clamp, a cell-attached patch clamp, an inside-out patch clamp, and an outside-out patch clamp between the recording electrode and the target neuron.

According to some embodiments, the system further comprises an optical fiber, wherein the at least one light source is coupled to the hollow glass tip by the optical fiber.

According to some embodiments, the at least one light source comprises one or more of a pulsed laser and a modulated laser. According to additional embodiments, the at least one light source comprises one or more of a neodymium-doped yttrium aluminum garnet laser and a titanium-sapphire laser.

According to some embodiments, the at least one light source is configured to emit light at a plurality of wavelengths.

According to some embodiments, the light sensor is an avalanche photodiode.

According to some embodiments, the system further comprises an amplifier configured to: receive the one or more photoacoustic signals from the ultrasound transducer; amplify the one or more photoacoustic signals to generate one or more amplified photoacoustic signals; and communicate the amplified photoacoustic signals to the processor, wherein the first set of signals comprises the amplified photoacoustic signals.

According to some embodiments, moving the hollow glass tip by a first increment in the one or more degrees of freedom comprises moving the hollow glass tip in the one or more degrees of freedom to increase one of an intensity of the one or more photoacoustic signals and an intensity of the one or more optical signals.

According to some embodiments, the instructions, when executed, further cause the processor to perform one or more second iterations, each second iteration comprising: receiving the electrical resistance measurements from the recording electrode; calculating, based on the electrical resistance measurements, the distance of the hollow glass tip with respect to the target neuron, and moving the hollow glass tip by a second increment in the one or more degrees of freedom via the actuator based on the distance. According to additional embodiments, moving the hollow glass tip by a second increment in the one or more degrees of freedom comprises moving the hollow glass tip in the one or more degrees of freedom to increase a value of the electrical resistance measurements from the recording electrode.

According to some embodiments, the ultrasound transducer is configured to detect the one or more photoacoustic signals across a range of at least 10 μm.

According to some embodiments, the light sensor is configured to detect the one or more optical signals across a range of at least 10 μm.

According to some embodiments, the target neuron is configured to emit the one or more optical signals based on genetic labeling of the target neuron.

A system for navigating to a target neuron is also provided. The system comprises a recording electrode including: a pipette having a hollow glass tip, and a headstage configured to detect electrical resistance measurements at the hollow glass tip; an actuator configured to move the hollow glass tip in one or more degrees of freedom; at least one light source coupled to the recording electrode and configured to emit light from the hollow glass tip; an ultrasound transducer configured to detect one or more photoacoustic signals in response to the light; a processor; and a non-transitory, computer-readable medium storing instructions that, when executed, cause the processor to: perform one or more first iterations, each first iteration comprising: receiving a set of signals associated with the one or more photoacoustic signals from the ultrasound transducer, calculating, based on the set of signals, a distance of the hollow glass tip with respect to the target neuron, responsive to a determination that the distance is greater than a predetermined threshold, moving the hollow glass tip by a first increment in the one or more degrees of freedom via the actuator based on the distance, and responsive to a determination that the distance is less than or equal to the predetermined threshold, maintaining the position of the hollow glass tip with respect to the target neuron via the actuator.

According to some embodiments, the instructions, when executed, further cause the processor to perform one or more second iterations, each second iteration comprising: receiving the electrical resistance measurements from the recording electrode; calculating, based on the electrical resistance measurements, the distance of the hollow glass tip with respect to the target neuron, and moving the hollow glass tip by a second increment in the one or more degrees of freedom via the actuator based on the distance.

A system for navigating to a target neuron is also provided. The system comprises a recording electrode including: a pipette having a hollow glass tip, and a headstage configured to detect electrical resistance measurements at the hollow glass tip; an actuator configured to move the hollow glass tip in one or more degrees of freedom; at least one light source coupled to the recording electrode and configured to emit light from the hollow glass tip; a light sensor configured to detect one or more fluorescence signals in response to the light; a processor; and a non-transitory, computer-readable medium storing instructions that, when executed, cause the processor to: perform one or more first iterations, each first iteration comprising: receiving a set of signals associated with the one or more fluorescence signals from the light sensor, calculating, based on the set of signals, a distance of the hollow glass tip with respect to the target neuron, responsive to a determination that the distance is greater than a predetermined threshold, moving the hollow glass tip by a first increment in the one or more degrees of freedom via the actuator based on the distance, and responsive to a determination that the distance is less than or equal to the predetermined threshold, maintaining the position of the hollow glass tip with respect to the target neuron via the actuator.

According to some embodiments, the instructions, when executed, further cause the processor to perform one or more second iterations, each second iteration comprising: receiving the electrical resistance measurements from the recording electrode; calculating, based on the electrical resistance measurements, the distance of the hollow glass tip with respect to the target neuron, and moving the hollow glass tip by a second increment in the one or more degrees of freedom via the actuator based on the distance.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts a conventional autopatching system for performing “neuron hunting” in accordance with an embodiment.

FIG. 2 depicts an illustrative fluoro-acoustic navigation system for a patch clamp electrode in accordance with an embodiment.

FIG. 3 depicts a flow diagram of an illustrative automated real-time feedback process for neuron hunting in accordance with an embodiment.

FIG. 4 illustrates a block diagram of an illustrative data processing system in which aspects of the illustrative embodiments are implemented in accordance with an embodiment.

FIG. 5 depicts exemplary experimental results of imaging performed using a 50 μm dimeter optical fiber probe in accordance with an embodiment.

FIG. 6 depicts exemplary fluorescence and photoacoustic feedback results received through a traditional glass microelectrode pipette in accordance with an embodiment.

FIG. 7 depicts an exemplary evaluation of resistance and feedback sensitivity as a function of a proximity to a probe tip in accordance with an embodiment.

FIG. 8 depicts an exemplary reconstructed image based on photoacoustic and fluorescence feedback in accordance with an embodiment.

FIG. 9 depicts an exemplary automated approach to a fluorescent bead and a stained cell based on fluorescence feedback intensity in accordance with an embodiment.

FIG. 10 depicts: (a) an exemplary optical architecture for automated fluorescence guidance in accordance with an embodiment; (b) an exemplary micropipette architecture and beam profile for automated fluorescence guidance in accordance with an embodiment; and (c) autonomous tapered fiber placement and neuronal approach in accordance with an embodiment.

FIG. 11 depicts: (a) a microscope image of a fluorescent bead illuminated through a tapered optical fiber during a raster scan in accordance with an embodiment; (b) a reconstruction of photon counts acquired from the region of interest shown in (a) in accordance with an embodiment; and (c) measurements and normalized data of the region of interest shown in (a) in accordance with an embodiment.

FIG. 12 depicts: (a) an overlayed image of brightfield and fluorescence images of cultured B35 neuroblastoma cells in accordance with an embodiment; (b) a reconstruction of photon counts acquired from the region of interest shown in (a) in accordance with an embodiment; and (c) measurements and normalized data of the region of interest shown in (a) in accordance with an embodiment.

FIG. 13 depicts the results of an automated approach toward fluorescent beads in accordance with an embodiment.

FIG. 14 depicts the results of an automated neuronal approach towards fluorescently labeled cells in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

All percentages, parts and ratios are based upon the total weight of the topical compositions and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “tissue” refers to any aggregation of similarly specialized cells which are united in the performance of a particular function.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As discussed herein, patch-clamp electrophysiology has been necessary for understanding both intercellular and intracellular processes of neurons through high resolution electrical recordings. This technique may enable study of healthy neuronal circuits as well as circuits implicated in neurological disease. However, this technique has a major barrier to entry due to the training required, low throughput, and dependence on the skill of the electrophysiologist. For example, navigation of patch clamp electrodes to target neurons is a limiting factor due to the amount of labor and skill level involved in localizing to neurons in vivo. Recent attempts have been made to circumvent these challenges with the advent of automated micropipette-based patch-clamp systems capable of neuronal targeting, recording, and more recently, micropipette reloading. However, these systems remain limited in their ability to target specific neuronal subtypes within the intact brain, where regions of interest are located beyond the imaging depth of current optical microscopy approaches.

The main distinction between different automated patch-clamp systems are the feedback mechanisms used for neuronal targeting, which include blind and image-guided systems. Blind systems utilize impedance measurements, caused by physically blocking current flow through the aperture of the micropipette tip to determine proper placement on the neuron. Though this technique has proven useful for providing feedback regarding the distance between the micropipette tip and the neuron, it is limited by its inability to target specific neuronal subtypes. To achieve real-time subtype-specific targeting, image-guided systems often leverage the use of fluorescent labels and dyes. These systems utilize high-powered optical imaging techniques, including confocal and two-photon (2P) microscopy, to iteratively locate the neuron and micropipette tip. This approach enables both cellular subtype targeting within heterogeneous environments and the correlation of electrical characteristics to neuronal morphology. Existing in vivo image-guided systems can track cells using fluorescence, but are usually limited to superficial brain regions, such as the uppermost cortical layers where the neurons and the micropipette tip can be reliably resolved.

An automated system whose feedback directly reports the distance between the neuron and micropipette tip (i.e., blind systems), while also allowing for the minimally invasive local detection of fluorescently labeled neurons (i.e., image-guided systems), will offer an improved approach. Such a system would require fluorescence excitation and emission collection at the micropipette tip, enabling a technology that is independent of a microscope.

Optical waveguides have been employed to circumvent limitations of imaging depth in several areas of neuroscience. Insertion of waveguides deep into tissue has enabled controlled light delivery, which has been leveraged for a variety of applications including imaging, optogenetics, and photometry. Optical detection and manipulation through waveguides combined with direct electrophysiological voltage measurements has been successfully achieved in several ways. However, optical waveguides often suffer from issues with detection sensitivity and spatial resolution. It would be advantageous to have an optical waveguide that enables both concentric and proximal measurements by implementing optical and electrophysiological alignment.

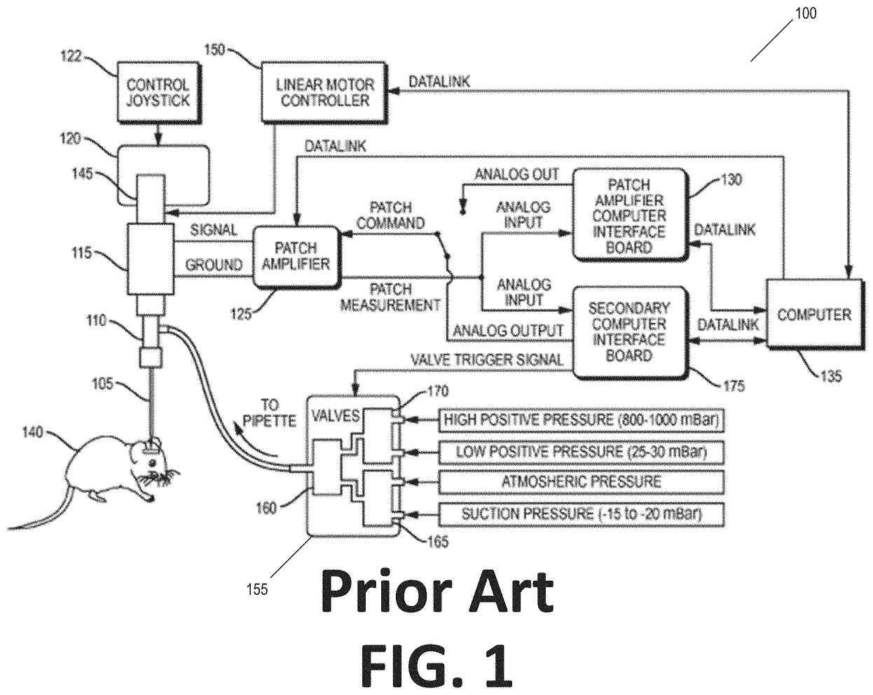

Referring now to FIG. 1, a conventional autopatching system for performing “neuron hunting” is depicted. A conventional autopatching system 100 may comprise a conventional in vivo patch setup, including a pipette 105, pipette holder 110, headstage 115, 3-axis linear actuator 120, control joystick 122, patch amplifier 125, patch amplifier computer interface board 130, and computer 135. The system may be configured for patching on headfixed mouse 140 or in another in vivo application. The autopatching system 100 may further comprise a programmable linear motor 145 and linear motor controller 150 for controlling up and down movement of pipette 105, a bank 155 of pneumatic valves 160, 165, and 170 for pressure control, and a secondary computer interface board 175 for controlling the linear motor 145 based on pipette resistance measurements. In some embodiments, the vertical axis of 3-axis linear actuator 120 is computer controlled, and the programmable linear motor 145 with linear motor controller 150 may be omitted. In some embodiments, the patch amplifier 125 and patch amplifier computer interface board 130 provide direct access to measurements and thus the secondary computer interface board 175 can be omitted. The computer 135 may receive resistance measurements and determine, based on the resistance measurements, whether the pipette 105 has reached a target neuron. Accordingly, the computer 135 and the linear motor control 150 may instruct the linear motor 145 and/or the linear actuator 120 to incrementally reposition the pipette 105 until a target neuron is reached. Additional components may be modified, replaced, and/or consolidated as would be apparent to a person having an ordinary level of skill in the art based on existing comparable parts, software, and implementation methodologies that are known in the field.

As generally described herein, autopatching systems such as the system 100 conventionally rely on resistance measurements to identify and incrementally move to locations of target neurons. However, resistance measurements may only be reliable for identifying the location of a target neuron at close range, e.g., 10 μm or less. Further, current image-guided navigation techniques require costly externally located microscopy equipment (e.g., DIC, confocal microscopy, and/or two-photon microscopy) and are only functional up to depths of 500 μm to 1 mm in tissue due to optical scattering.

Ideally, a navigation tool with a larger feedback range would allow for neuron hunting at greater depths and with greater precision, opening the possibility for high-resolution studies of deep neural cells. Accordingly, the navigation tool may enable study of neuronal signals deep in the living brain with high spatial resolution and genetic specificity to understand complex neural interactions. Further, the navigation tool may be utilized to precisely target neurocircuits (e.g., multiple neurons along a signal pathway) known to be activated in a variety of conditions or disease states.

### Fluoro Acoustic Navigation System for Patch Clamp Electrophysiology

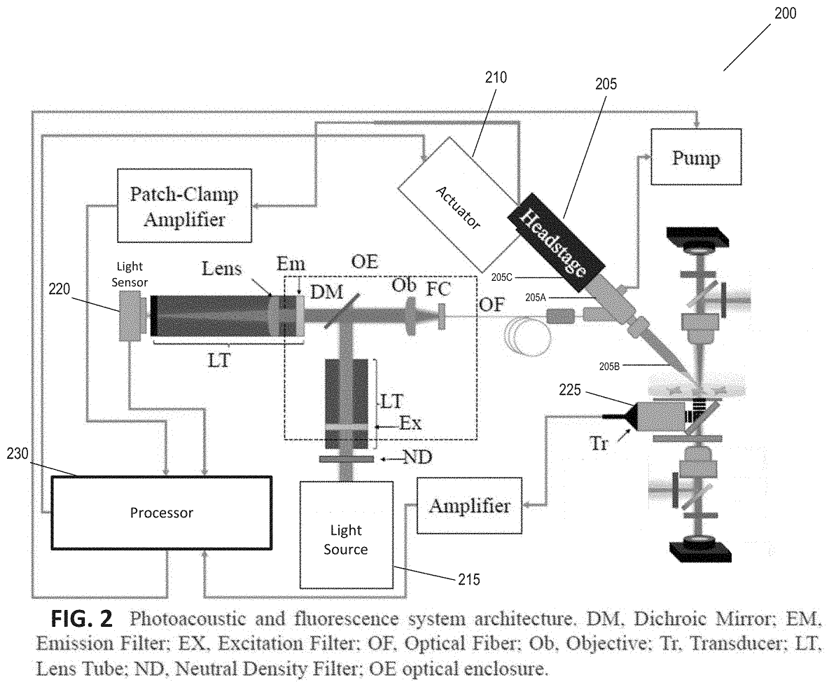

Referring now to FIG. 2, an illustrative fluoro-acoustic navigation system 200 for a patch clamp electrode is depicted in accordance with an embodiment. As shown in FIG. 1, the system 200 may comprise a recording electrode 205, an actuator 210, a light source 215, a light sensor 220, an ultrasound transducer 225, and a processor 230.

In some embodiments, the recording electrode 205 comprises a pipette holder 205A including a pipette with a hollow glass tip 205B at a distal end thereof. The hollow glass tip 205B may form an interior cavity configured to hold a solution, e.g., an electrolyte solution. In some embodiments, the solution may resemble an extracellular solution and/or cytoplasm, depending on the recording mode. The solution may provide higher conductivity from the hollow glass tip 205B to the interior of a cell in contact therewith. In some embodiments, the hollow glass tip 205B may taper or narrow from a proximal end to a distal end thereof. In some embodiments, the proximal end of the hollow glass tip 205B has a diameter of about 1 mm. However, the diameter of the proximal end may be 5 mm, 4 mm, 3 mm, 2 mm, 1 mm, 500 μm, less than 500 μm, or individual values or ranges therebetween. In some embodiments, the distal end of the hollow glass tip 205B has a diameter of about 1 μm. However, the diameter of the distal end may be 3 μm, 2 μm, 1 μm, 500 nm, less than 500 nm, or individual values or ranges therebetween. Additional sizes and configurations of the hollow glass tip 205B are contemplated herein as would be apparent to a person having an ordinary level of skill in the art. The recording electrode may further comprise a headstage 205C in electrical communication with an interior of the hollow glass tip 205B. The headstage 205C may comprise built-in circuitry to transmit electrical signals between the hollow glass tip 205B and other components of the fluoro-acoustic navigation system 200, e.g., for recording resistance measurements. While an exemplary recording electrode 205 is described and depicted, it is contemplated that the recording electrode 205 may include additional or alternative components conventionally used for recording electrodes in the field of patch clamp electrophysiology as would be known to a person having an ordinary level of skill in the art.

Referring once again to FIG. 2, the light source 215 is a laser. For example, the light source 215 may be a high-intensity laser, such as pulsed laser or a modulated laser (e.g., a nanosecond laser). In some embodiments, the laser is a neodymium-doped yttrium aluminum garnet (Nd:YAG) laser. In some embodiments, the laser is a titanium-sapphire (TiSa) laser. In some embodiments, the laser may be configured to provide fast excitation and a resultant photoacoustic signal. In another example, the light source may be a continuous wave laser. In some embodiments, the continuous wave laser may be configured to emit light at a continuous power level to generate a resultant photoacoustic signal. In some embodiments, the continuous wave laser may be configured to emit light at a modulated level to generate a resultant photoacoustic signal. In some embodiments, the laser is configured to emit light from the hollow glass tip 205B. For example, the light source may be coupled to the hollow glass tip 205B via an optical fiber to deliver the light from the light source 215 to the hollow glass tip 205B.

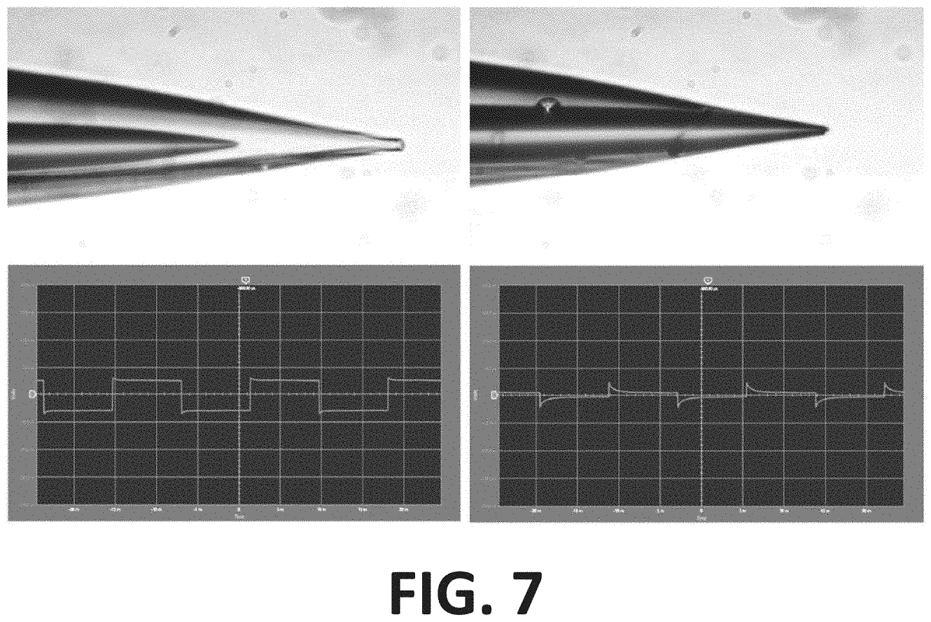

In some embodiments, the optical fiber may be a tapered optical fiber (e.g., as depicted in FIG. 7). A tapered optical fiber may be selected based on a diameter of the tip of the optical fiber in order to match the diameter of the hollow glass tip 205B. In some embodiments, the tapered optical fiber may be placed with respect to the hollow glass tip 205B based on current feedback. For example, resistance through the tapered optical fiber may increase as the tapered optical fiber approaches the hollow glass tip 205B. Accordingly, current through the tapered optical fiber may be measured and used to assess a distance of the tapered optical fiber from the end of the hollow glass tip 205B in order to ensure proper fiber placement. As the tapered optical fiber is advanced, current measurement may decrease due to increased resistance until the current measurement is zero or near zero, thereby indicating proper placement of the tapered optical fiber at the end of the hollow glass tip 205B. In some embodiments, a micromotor or other powered component may be used to advance the tapered optical fiber based on the current measurements.

Furthermore, the light sensor 220 is configured to detect a light response signal at the hollow glass tip 205B and communicate the light response signal to the processor 230. For example, the signal may comprise a wavelength of detected light and/or an intensity of detected light. In some embodiments, the light sensor 220 may be a photodiode such as an avalanche photodiode. In some embodiments, the light sensor 220 may be coupled to the hollow glass tip 205B via an optical fiber to detect light at the hollow glass tip 205B.

Similarly, the ultrasound transducer 225 may be configured to detect a photoacoustic signal at the hollow glass tip 205B and communicate the signal to the processor 230. In some embodiments, the ultrasound transducer 225 may be a high-frequency ultrasound transducer, e.g., 10 MHz. As depicted, the ultrasound transducer 225 may be separate from the recording electrode 205. However, in some embodiments, the ultrasound transducer 225 may be coupled to the recording electrode 205 at a portion of the hollow glass tip 205B proximate to the distal end. For example, the ultrasound transducer 225 may be within the interior cavity of the hollow glass tip 205B. The ultrasound transducer 225 may be positioned sufficiently close to the distal end of the hollow glass tip 205B to minimize attenuation of the photoacoustic signal sensed from the distal end of the hollow glass tip 205B. The distance of the ultrasound transducer 225 from the distal end of the hollow glass tip 205B may be 0.5 cm, 1 cm, 2 cm, 3 cm, 4 cm, 5 cm, greater than 5 cm, or individual values or ranges therebetween. However, in some embodiments, a distance of less than 0.5 cm may be implemented.

In some embodiments, the light source 215 and the light sensor 220 may be combined and/or consolidated as an optical unit comprising any number of additional components. For example, as shown in FIG. 2, the light source 215 and light sensor 220 may be included in an optical enclosure. The light source 215 may be arranged in the optical enclosure with one or more of a neutral density filter, a lens tube, an excitation filter, a dichroic mirror, an objective, and/or a fiber connector to transmit the light from the light source 215 to the hollow glass tip 205B. Further, the light sensor 220 may be arranged in the optical enclosure with a lens tube, an emission filter, and the dichroic mirror to detect light from the hollow glass tip 205B. However, the light source 215 and the light sensor 220 may be arranged with a variety of optical components as would be apparent to one having an ordinary level of skill in the art to emit light to the hollow glass tip 205B and to receive light therefrom.

In some embodiments, one or more parameters of the light emitted by the light source 215 may be adjusted. In some embodiments, the intensity of light may be adjusted. In some embodiments, the frequency of light emission may be adjusted. In some embodiments, the color or wavelength of light may be adjusted. In some embodiments, the parameters of the emitted light may be fixed. Some parameters of the emitted light (e.g., intensity) may control the feedback range of the system 200, i.e., the distance at which appropriate real-time feedback may be acquired by the light sensor 220 and/or ultrasound transducer 225 to perform necessary calculations. In some embodiments, the feedback range is about 20 μm. However, the feedback range may be 40 μm, 35 μm, 30 μm, 25 μm, 20 μm, 15 μm, 10 μm, less than 10 μm, or individual values or ranges therebetween. While conventional autopatching methods utilizing resistance feedback may provide a feedback range of less than 10 μm, the system 200 may be configured to operate over a larger range as described herein, thereby facilitating a greater success rate during neuron hunting.

Referring once more to FIG. 2, the actuator 210 is configured to move the hollow glass tip 205B in one or more degrees of freedom with respect to a tissue. In some embodiments, the actuator 210 may be a micromanipulator. In some embodiments, the actuator 210 comprises one or more motors, e.g., linear motors; however, any actuating components may be used as would be known to a person having an ordinary level of skill in the art. In some embodiments, the actuator 210 is a 3-axis linear actuator configured to translate the hollow glass tip 205B with respect to three axes. However, in some embodiments, the actuator 210 may be configured to translate the hollow glass tip 205B with respect to less than three axes, e.g., one or two axes. In some embodiments, translation with respect to the remaining axes may be locked. Further, in some embodiments, the actuator 210 may be configured to rotate the hollow glass tip 205B with respect to one, two, or three axes, which may be coincident with any of the translational axes. In some embodiments, rotation with respect to any number of rotational axes may be locked. Accordingly, the actuator 210 may be configured to move the hollow glass tip 205B with respect to the tissue in up to six degrees of freedom. In some embodiments, movement of the hollow glass tip 205B by the actuator 210 is computer controlled, e.g., by the processor 230. In some embodiments, the processor 230 or another computing device controls movement of the hollow glass tip 205B in each available degree of freedom. However, the processor 230 may control movement in less than all available degrees of freedom. For example, one or more degrees of freedom may be controlled by the processor while additional degrees of freedom may be controlled by a user manually and/or through a user input device, e.g., a joystick.

In some embodiments, the processor 230 may instruct movement of the hollow glass tip 205B by the actuator 210 according to an automated feedback process in order to carry out “neuron hunting.” The automated feedback process may be an iterative process comprising incremental movements based on real-time fluorescence feedback from the light sensor 220 and/or real-time photoacoustic feedback from the ultrasound transducer 225, i.e., fluoro-acoustic navigation.

To initiate the process, a tissue sample may be positioned with respect to the system 200 proximate the recording electrode 205. The tissue may be selected based on the requirements or protocols of a study, e.g., a target neuron. For example, the system 200 may be utilized with an animal to perform in vivo experiments. In another example, the system 200 may be utilized with a tissue sample to perform in vitro experiments. In some embodiments, the system 200 may be utilized to target neurons in brain tissue. However, the system 200 may be utilized to target neurons in other tissues, including but not limited to central nervous system tissue or peripheral nervous system tissue. Furthermore, the system 200 may be utilized to target additional types of cells that exhibit action potentials or other measurable changes in membrane potential, e.g., cardiomyocytes that exhibit cardiac action potentials.

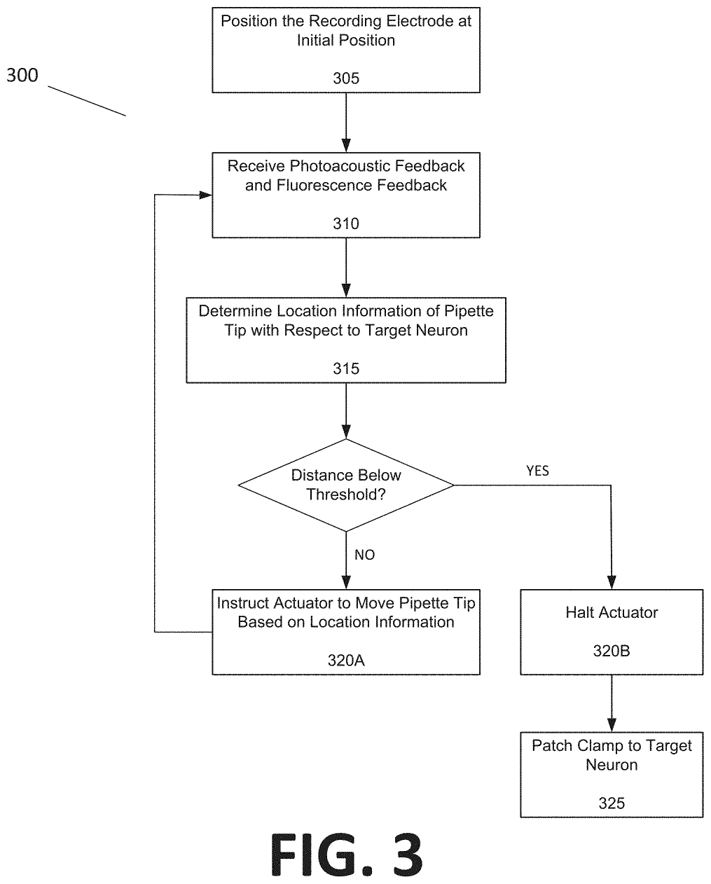

Referring now to FIG. 3, a flow diagram of an illustrative automated real-time feedback process for neuron hunting is depicted in accordance with an embodiment. The feedback process 300 may be carried out by a system such as fluoro-acoustic navigation system 200 of FIG. 2. The pipette tip (i.e., hollow glass tip) of the recording electrode is positioned 305 at an initial position in a tissue. The pipette tip may be placed at an initial position based on the target neuron. For example, the pipette tip may be positioned 305 at an approximate depth of the target neuron and/or in a section of brain tissue expected to contain the target neuron. In some embodiments, the positioning 305 is completed manually by a user. In some embodiments, the positioning 305 is completed by user input from a user input device to direct the movement of the recording electrode. In some embodiments, the processor may position 305 the pipette tip based on location information related to the target neuron and a known spatial relationship between the pipette tip and the tissue.

Thereafter, the processor may perform one or more iterations comprising receiving 310 photoacoustic feedback and/or fluorescence feedback, determining 315 location information related to the target neuron, and instructing 320 the actuator based on the location information.

The processor may receive 310 photoacoustic feedback from the ultrasound transducer and/or fluorescence feedback from the light sensor in response to light emitted from the light source. In some embodiments, the processor instructs the light source to emit light at a known time and/or interval and associates the received feedback with the known parameters of the emitted light. In some embodiments, the light source may be controlled by a separate processor.

In some embodiments, the photoacoustic feedback and/or fluorescence feedback may include a natural response of the target neuron to the emitted light. In some embodiments, the photoacoustic feedback and/or fluorescence feedback may include a modified response of the target neuron to the emitted light. For example, the target neurons may have been modified or labeled to have a specific response to an emitted light by genetic encoding, exogenous contrast, optogenetics, and/or other known methods of modifying photoacoustic and/or fluorescence responses of cells. For example, the target neurons may be modified to have increased sensitivity in order to improve detection and/or to have a response at a narrow wavelength range in order to increase specificity. Subjects and/or samples with genetically modified or labeled neurons as discussed herein are commercially available and generally known to a person having an ordinary level of skill in the art.

Based on the photoacoustic feedback and/or fluorescence feedback, the processor determines 315 location information related to the target neuron. For example, the processor may approximate a distance and/or directional location of the target neuron with respect to the pipette tip. In some embodiments, the processor may identify a known response of the target neuron from the photoacoustic feedback and/or fluorescence feedback. For example, based on the frequency, wavelength, and other parameters of the probing radiation (i.e., the emitted light), the target neurons may have a known response. For example, the target neuron may produce a signature response, i.e., a signature acoustic wave and/or light wave in response to the emitted light. A signature response may be defined by characteristic values or ranges of values for one or more parameters of the resulting wave including but not limited to a characteristic amplitude, a characteristic frequency, and/or a characteristic time delay (e.g., indicative of distance of the target neuron). In some embodiments, the processor may identify a characteristic parameter or combination of characteristic parameters unique to the target neuron in order to identify the target neuron based on the photoacoustic feedback. Therefore, in some embodiments, the processor may identify and extract feedback information associated with the target neuron from the photoacoustic feedback and/or fluorescence feedback. In some embodiments, the feedback information associated with the target neuron comprises a portion of the photoacoustic feedback and/or fluorescence feedback at predetermined wavelengths. In some embodiments, the target neuron may have a known response profile. For example, the target neuron may have a varying response to different wavelengths of emitted light to form a signature profile. A signature profile may be defined by a signature response to each of a plurality of emitted wavelengths of light. In some embodiments, the signature response may comprise raw values or ranges of values for one or more parameters as described. In some embodiments, the signature response may comprise relative values or ranges of values for the one or more parameters. For example, the amplitude of the response at each wavelength may form a curve, thereby indicating a peak response amplitude for a known wavelength of emitted light as part of the signature profile. Therefore, feedback may be recorded in response to a plurality of emitted wavelengths of light, and the feedback may be used to identify and extract feedback information associated with the target neuron based on the known response profile. Accordingly, the processor may use the feedback information to determine 315 location information including distance information and/or directional information.

In some embodiments, the location information includes an estimated distance of the target neuron from the pipette tip. For example, the intensity of the photoacoustic feedback and/or fluorescence feedback may correspond to a distance of the target neuron. Accordingly, a distance of the target neuron may be calculated by the system based on the feedback during each iteration and may be utilized for instructing movement of the pipette tip during step 320A as further described herein. However, in some embodiments, the intensity of the photoacoustic feedback and/or fluorescence feedback may only indicate a relative distance of the target neuron. For example, by comparing the feedback during a current iteration with the feedback from a previous iteration, the processor may determine whether the distance to the target neuron was increased or decreased by the preceding movement of the pipette tip during step 320A of the previous iteration. Accordingly, the relative distance of the target neuron may be determined by the system during each iteration. In such embodiments, prior to performing the one or more iterations, the processor may initially instruct a series of “blind” movements of the pipette tip to collect initial feedback information for comparison during subsequent iterations. For example, the system may perform one or more blind movements along each axis of movement.

The processor may assess whether the distance is above or below a predetermined threshold and instruct 320 the actuator based on the distance to the target neuron. The instruction may vary based on whether the distance is above or below the predetermined threshold.

If the distance is above the predetermined threshold, the system may instruct 320A the actuator to move the pipette tip based on the location information. The movement instruction may comprise a direction and a movement distance (e.g., a vector) that, based on the location information, moves the pipette tip closer to the target neuron. In some embodiments, the movement distance is an incremental, predetermined distance. In some embodiments, the movement distance may be constant across all iterations. In some embodiments, the movement distance may vary based on the intensity of the photoacoustic feedback and/or fluorescence feedback. For example, the intensity values or ranges of intensity values may correspond to predetermined movement distances. Accordingly, as feedback intensity increases (i.e., indicating decreasing distance from the target neuron), the corresponding movement distance may decrease. In some embodiments, the distance may be determined in each iteration based on a calculated distance of the target neuron. As shown in FIG. 3, upon completion of step 320A, a subsequent iteration may be initiated by returning to step 310 of receiving photoacoustic feedback and/or fluorescence feedback at the new position.

Alternatively, where the distance is below the predetermined threshold, the processor may halt 320B the actuator, thereby maintaining the position of the pipette tip from the previous iteration. In some embodiments, the processor affirmatively instructs 320B the actuator to maintain the position (i.e., a null instruction). In some embodiments, the processor halts 320B the actuator by not providing a further movement instruction. Where the distance is below the predetermined threshold, the distance to the target neuron may be sufficiently small to carry out the remainder of the process and therefore cease performance of the iterations. Therefore, step 320B is performed only in the final iteration.

After performing the one or more iterations, the processor comprises patch clamping 320 to the target neuron. In some embodiments, the predetermined threshold is a distance sufficiently small to allow for patch clamping by conventional methods. Thus, patch clamping 320 may comprise various steps associated with known procedures for cell recording. For example, the patch clamping may comprise whole-cell recording procedures. Accordingly, patch clamping 320 to the target neuron comprises forming a gigaohm seal and rupturing the cell membrane via suction. In some embodiments, the gigaohm seal may provide a resistance in the range of about 1 to about 1000 gigaohms (i.e., on the order of a gigaohm). In some embodiments, the gigaohm seal may provide a resistance in the range of about 10 to about 100 gigaohms. Additional ranges are contemplated herein as would be apparent to a person having an ordinary level of skill in the art.

However, the process 300 may be used for localization for other cell recording methods. In another example, the patch clamping may comprise outside-out recording. Accordingly, patch clamping 320 comprises similar steps to the whole-cell recording method and further comprises retracting the pipette after rupturing the membrane, thereby detaching a portion of the membrane on the pipette tip and forming a small vesicular structure for use in cell recording. In another example, the patch clamping may comprise cell-attached recording. Accordingly, patch clamping 320 comprises applying sufficiently mild suction to form a gigaohm seal without rupturing the cell membrane. In another example, the patch clamping may comprise inside-out recording. Accordingly, patch clamping 320 comprises similar steps to the cell-attached recording method and further comprises retracting the pipette after formation of the gigaohm seal, thereby detaching a portion of the membrane and exposing the cytosolic surface to air. By any of the described methods of patch clamping 320, the system is thereby prepared for cell recording of the target neuron.

In some embodiments, the predetermined threshold may vary based on the method of cell recording. For example, the predetermined threshold may be a distance at which sufficiently strong suction may be applied to form a gigaohm seal as is common in whole cell recording and outside-out recording methods. In another example, the predetermined threshold may be a distance at which sufficiently mild suction may be applied to form a gigaohm seal without rupturing the cell membrane as is common in cell-attached recording and inside-out recording methods. Accordingly, in some embodiments, the predetermined threshold may be 1 μm, 500 nm, 250 nm, 100 nm, less than 100 nm, or individual ranges or values therebetween.

In some embodiments, the predetermined threshold is a distance from the target neuron at which other conventional methods of localizing to the target neuron may be used to complete the positioning of the recording electrode (i.e., a “close range mode”). Accordingly, the process 300 may further comprise completing localization to the target neuron at close range by conventional methods. For example, when the pipette tip is in close range of the target neuron, the system may rely on resistance measurements from the recording electrode to approximate a distance and/or location of the target neuron in the conventional manner. Accordingly, the processor may instruct incremental movements of the pipette tip until the pipette tip contacts the target neuron and/or is positioned sufficiently close to the target neuron to initiate patch clamping. Accordingly, in some embodiments, the predetermined threshold may be 10 μm, 9 μm, 8 μm, 7 μm, 6 μm, 5 μm, 4 μm, 3 μm, 2 μm, 1 μm, less than 1 μm, or individual ranges or values therebetween.

In additional embodiments, completing localization to the target neuron in close range mode may be performed by reducing the intensity of the emitted light. In some embodiments, reducing the intensity of the emitted light decreases the range of the emitted light. Accordingly, photoacoustic and/or fluorescence feedback from longer range is eliminated and only feedback from cells at close range is received. In some embodiments, the emitted light in close range mode has a feedback range of 10 μm, 9 μm, 8 μm, 7 μm, 6 μm, 5 μm, 4 μm, 3 μm, 2 μm, 1 μm, 500 nm, 250 nm, 100 nm, less than 100 nm, or individual ranges or values therebetween. As such, in some embodiments, the predetermined threshold may be coincident with the feedback range of the emitted light in close range mode. In some embodiments, the range of the emitted light may be consistently or intermittently reduced throughout the entire process 300 as the pipette tip closes in on the target neuron.

In some embodiments, completing localization to the target neuron in close range mode may be performed by calculating the travel time of sound waves. For example, where an ultrasound transducer is located on a portion of the pipette tip, the processor may determine a distance that a feedback sound wave (i.e., a photoacoustic signal) traveled based on the time that the sound wave is received with respect to the time of light emission. The travel time may be determined based on sound wave propagation properties and the distance of the ultrasound transducer from the hollow glass tip (i.e., the distance the wave must travel up through the glass material). Accordingly, the distance of a target neuron may be determined at close range based on sound wave travel time. In some embodiments, sound wave travel time may be utilized when the target neuron is within a range of 10 μm, 9 μm, 8 μm, 7 μm, 6 μm, 5 μm, 4 μm, 3 μm, 2 μm, 1 μm, 500 nm, 250 nm, 100 nm, less than 100 nm, or individual ranges or values therebetween. As such, in some embodiments, the predetermined threshold may be coincident with the range for calculating target neuron distance based on sound wave travel time in close range mode.

In some embodiments, the process 300 may be carried out by a processor or other computing device as described herein (e.g., the system 200 of FIG. 2) using a software algorithm. In some embodiments, the software may collect fluorescence and/or photoacoustic feedback by scanning a predetermined area of interest defined by the range of feedback as described herein. For example, the software may instruct raster scanning. Based on the collected feedback, the software may instruct repositioning of the pipette tip as described herein based on the algorithm. In some embodiments, the software may identify a position corresponding to a peak feedback signal (e.g., intensity of the signal or amplitude of the signal) from the scan. At the new position, the software may perform another scan and instruct further repositioning. The software may repeat the scanning and repositioning until a threshold peak feedback signal is obtained and/or a threshold distance from the target neuron is calculated as described. Thereafter, the software may reduce the scanning area to a smaller range and scan and reposition until a second threshold peak feedback signal is obtained and/or a second threshold distance from the target neuron is calculated. In some embodiments, the software may performing one or more additional sequences of reducing scanning area and performing scanning and repositioning as described.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art

The devices, systems, and methods described herein may further comprise any number of components known and/or used in conventional autopatching systems. FIG. 2 illustrates some such additional components. For example, in some embodiments, the system may comprise an amplifier configured to amplify photoacoustic signals detected by the ultrasound transducer. Accordingly, the photoacoustic signals may be amplified to generate amplified photoacoustic signals that are received by the processor for use in the various calculations. In another example, the system may comprise a syringe pump and/or suction device for applying negative pressure to the target neuron to complete patch clamping. In another example, the system may comprise a patch clamp amplifier configured to amplify the voltage measurements received from the recording electrode during neuron hunting and/or patch clamp electrophysiology. Additional or alternative components may be incorporated into the devices and systems described herein as would be apparent to one having an ordinary level of skill in the art.

While the processor 230 is generally described as directly interfacing with various components, the processor 230 may receive and transmit signals indirectly via additional components. In some embodiments, the processor 230 may receive and transmit signals through a data acquisition (DAQ) device or card. For example, the DAQ device may include a field-programmable gate array. In some embodiments, the processor 230 may receive photoacoustic and/or fluorescence feedback through a DAQ device and/or control a pump through the DAQ device. However, any communication associated with the processor as described herein may be facilitated through a DAQ device.

In some embodiments, a plurality of recording electrodes may be utilized to simultaneously perform patch clamp electrophysiology on a plurality of target neurons. In some embodiments, each recording electrode comprises an individual pipette having a pipette tip as described herein. In some embodiments, a multipipette may include a plurality of electrodes for simultaneously performing patch clamp electrophysiology on a plurality of target neurons. In some embodiments, the pipette tips of the multipipette may be individually movable. In some embodiments, a plurality of pipettes and/or pipette tips may be controlled by a single processor and/or actuator. As such the devices, systems, and methods described herein may be used to precisely target neurocircuits.

In some embodiments, the system 200 may not collect and/or utilize both photoacoustic feedback and fluorescence feedback. For example, the system may be configured to collect photoacoustic feedback and may not be configured to collect fluorescence feedback. Accordingly, the system 200 may omit components used solely for collecting fluorescence feedback. In some embodiments, photoacoustic feedback may be sufficient to perform neuron hunting without the use of fluorescence feedback.

In another example, the system 200 may be configured to collect fluorescence feedback and may not be configured to collect photoacoustic feedback. Accordingly, the system 200 may omit components used solely for collecting photoacoustic feedback. In some embodiments, fluorescence feedback may be sufficient to perform neuron hunting without the use of photoacoustic feedback.

In some embodiments, the system 200 may comprise a plurality of light sources and/or a light source configured to emit light at a plurality of wavelengths. Accordingly, the system 200 may be capable of collecting photoacoustic and/or fluorescence feedback in response to a plurality of wavelengths of light and associating each feedback signal with the corresponding parameters of emitted light. The processor may determine a photoacoustic and/or fluorescence feedback profile that may be used to differentiate between cell types, thereby assisting in identification of target neurons. In the same manner, the combination of photoacoustic feedback and fluorescence feedback may form a profile that that may be used to differentiate between cell types, thereby assisting in identification of target neurons.

In some embodiments, the system 200 may be configured to localize to specific portions of neuronal cells (e.g., soma, dendrite, axon). In some embodiments, one or more portions of the cell may produce unique responses or response profiles in the form of photoacoustic, fluorescence, or other feedback. The processor may thereby distinguish between the portions of the cells to instruct movement of the hollow glass tip.

While the described embodiments are discussed with respect to identifying and localizing to target neurons, the apparatuses, systems, and methods described herein may be adapted for other types of cells that exhibit a fluctuating membrane potential as would be apparent to a person having an ordinary level of skill in the art. For example, the apparatuses, systems, and methods may be adapted to identifying and localizing to cardiomyocytes to evaluate cardiovascular activity and health.

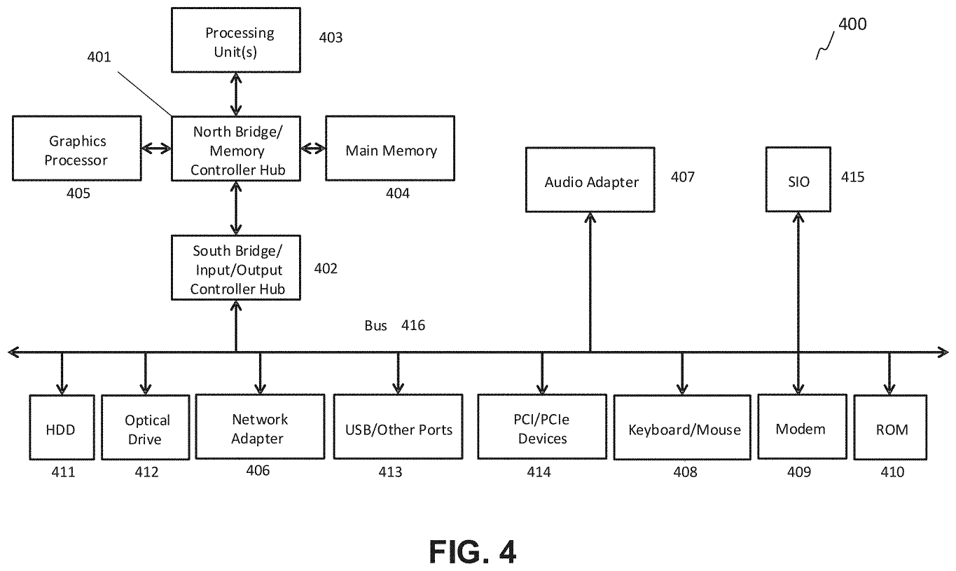

FIG. 4 illustrates a block diagram of an illustrative data processing system 400 in which aspects of the illustrative embodiments are implemented. The data processing system 400 is an example of a computer, such as a server or client, in which computer usable code or instructions implementing the process for illustrative embodiments of the present invention are located. In some embodiments, the data processing system 400 may be a server computing device. For example, data processing system 400 can be implemented in a server or another similar computing device operably connected to a system 200 as described above.

In the depicted example, data processing system 400 can employ a hub architecture including a north bridge and memory controller hub (NB/MCH) 401 and south bridge and input/output (I/O) controller hub (SB/ICH) 402. Processing unit 403, main memory 404, and graphics processor 405 can be connected to the NB/MCH 401. Graphics processor 405 can be connected to the NB/MCH 401 through, for example, an accelerated graphics port (AGP).

In the depicted example, a network adapter 406 connects to the SB/ICH 402. An audio adapter 407, keyboard and mouse adapter 408, modem 409, read only memory (ROM) 410, hard disk drive (HDD) 411, optical drive (e.g., CD or DVD) 412, universal serial bus (USB) ports and other communication ports 413, and PCI/PCIe devices 414 may connect to the SB/ICH 402 through bus system 416. PCI/PCIe devices 414 may include Ethernet adapters, add-in cards, and PC cards for notebook computers. ROM 410 may be, for example, a flash basic input/output system (BIOS). The HDD 411 and optical drive 412 can use an integrated drive electronics (IDE) or serial advanced technology attachment (SATA) interface. A super I/O (SIO) device 415 can be connected to the SB/ICH 402.

An operating system can run on the processing unit 403. The operating system can coordinate and provide control of various components within the data processing system 400. As a client, the operating system can be a commercially available operating system. An object-oriented programming system, such as the Java programming system, may run in conjunction with the operating system and provide calls to the operating system from the object-oriented programs or applications executing on the data processing system 400. As a server, the data processing system 400 can be an IBM® eServer™ System® running the Advanced Interactive Executive operating system or the Linux operating system. The data processing system 400 can be a symmetric multiprocessor (SMP) system that can include a plurality of processors in the processing unit 403. Alternatively, a single processor system may be employed.

Instructions for the operating system, the object-oriented programming system, and applications or programs are located on storage devices, such as the HDD 411, and are loaded into the main memory 404 for execution by the processing unit 403. The processes for embodiments described herein can be performed by the processing unit 403 using computer usable program code, which can be located in a memory such as, for example, main memory 404, ROM 410, or in one or more peripheral devices.

A bus system 416 can be comprised of one or more busses. The bus system 416 can be implemented using any type of communication fabric or architecture that can provide for a transfer of data between different components or devices attached to the fabric or architecture. A communication unit such as the modem 409 or the network adapter 406 can include one or more devices that can be used to transmit and receive data.

Those of ordinary skill in the art will appreciate that the hardware depicted in FIG. 4 may vary depending on the implementation. Other internal hardware or peripheral devices, such as flash memory, equivalent non-volatile memory, or optical disk drives may be used in addition to or in place of the hardware depicted. Moreover, the data processing system 400 can take the form of any of a number of different data processing systems, including but not limited to, client computing devices, server computing devices, tablet computers, laptop computers, telephone or other communication devices, personal digital assistants, and the like. Essentially, data processing system 400 can be any known or later developed data processing system without architectural limitation.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples:

### Examples

### Example 1: Initial Imaging Experiments with a 50 μm Dia. OF Probe

Methods: Initial imaging experiments were performed using a 50 μm dia., 0.22 NA optical fiber (OF) probe and either a 1000 W LED excitation source for FL-only signal generation or a ns-pulsed Nd:YAG/TiSa laser source operating at 480 nm for dual PA/FL signal generation. FL polyethylene spheres (PS) (125 μm dia.) were used as PA/FL targets and detected using either a focused 10 MHz ultrasound transducer or an avalanche photodiode (APD), respectively. In a separate set of experiments a 1 μm dia. Cr/Au coated tapered OF probe was coupled to a laser source operating at 480 nm or 532 nm for either FL imaging of PS targets or PA imaging of a Cr-coated Air Force target (AFT), respectively. In all imaging experiments, data acquisition and system synchronization were performed using a dual DAQ/FPGA module. A programmable micromanipulator mounted on the stage of an inverted microscope was used for probe positioning.

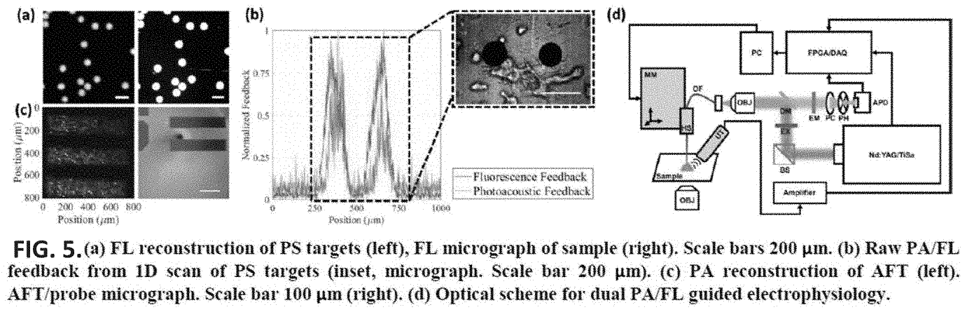

Results: Our results indicate that a 50 μm dia. OF probe can be used for continuous-wave FL imaging (FIG. 5 at (a)) up to 1 mm from the surface of PS targets, and two-way PA/FL signal generation and detection using a pulsed excitation source (FIG. 5 at (b)). Subsequent experiments using a 1 μm dia. tapered OF probe and 480 nm pulsed source, demonstrate FL signal generation and detection from PS targets. Experiments using the same tapered OF probe coupled to a 532 nm pulsed source demonstrate PA signal generation and detection from a Cr-coated AFT. A PA reconstruction of the AFT (left) and a micrograph of the probe above the AFT (right) is shown in FIG. 5 at (c). A generalized optical scheme for dual PA/FL guided electrophysiology is shown in FIG. 5 at (d).

Preliminary results demonstrate dual PA and FL signal generation and detection through the same 50 μm dia. OF probe using a pulsed source and PA/FL detection through a 1 μm dia. tapered OF probe. Future studies will test the electrical capabilities of integrated tapered OF/micropipette electrode assemblies.

### Example 2: Fluorescence and Photoacoustic Feedback Through a Traditional Glass Pipette

Methods: A photoacoustic micropipette (PMP) electrode capable of real-time targeting utilizing photoacoustic and fluorescence feedback was developed. The system (shown in FIG. 2) includes a tunable LS-2134-LT40 Nd:YAG/Ti: Sapphire nanosecond pulsed laser. The laser provides an excitation light with a full width half maximum of 12-15 ns at a pulse repetition rate of 10 Hz. An optical fiber inserted into the PMP allows for deposition and collection of light at the micropipette tip. A 10 MHz transducer inside a custom 3D printed housing is used to detect induced photoacoustic signals. Generated fluorescence is detected by an avalanche photodiode. Automated algorithms, developed in-house, are used to move the PMP in 3 dimensions.

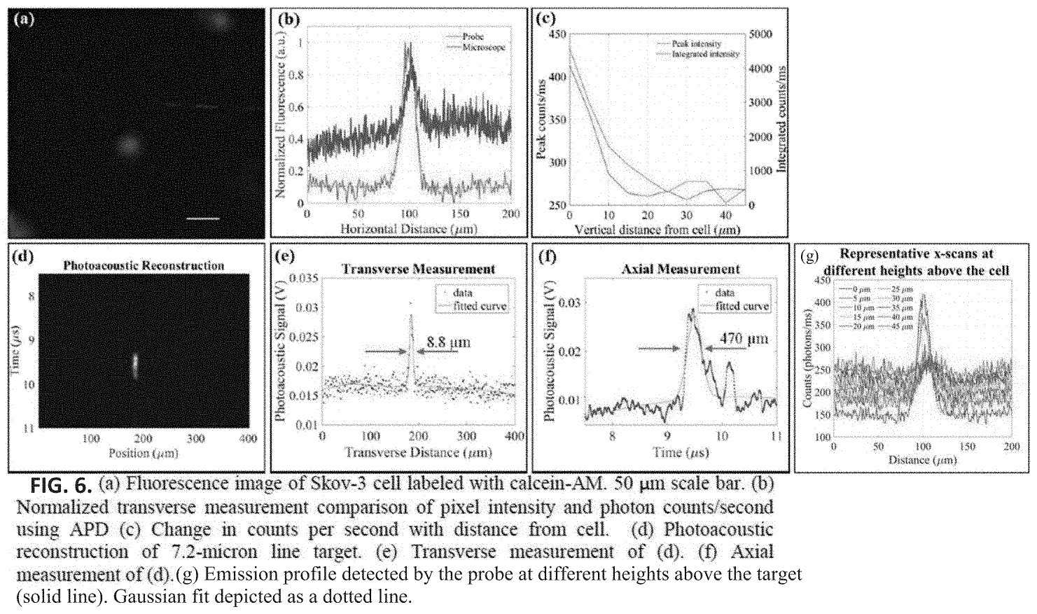

Results: Due to the material properties of traditional glass microelectrode pipettes, they can function as optical waveguides to both deliver excitatory light and detect emitted fluorescence from a spot size that is comparable to a cell. Results identify an increase in fluorescence feedback (FIG. 6 at (a)-(c) and (g)) and photoacoustic feedback (FIG. 6 at (d)-(f)) as the PMP tip is moved towards a target. Thus, providing a method of automated robotic movement of the PMP in the direction that maximizes feedback.

Introducing a navigational capability into micropipette electrodes will allow precise targeting in combination with high resolution recording of cells at depths beyond what is currently possible.

### Example 3: Internal Optics with Respect to Micropipette Tip

Methods: Internal optics in the form of a tapered fiber were advanced through a micropipette tip to determine sensitivity of feedback. Current was measured over time as the tapered fiber was advanced to assess the effect of the fiber position on resistance.

Results: Results identify an increase in resistance as the optics approach the tip, where feedback is most sensitive. FIG. 7 depicts a measurement of current through the tapered fiber at a first fiber position spaced from the tip and a second fiber position at the tip. As a result of the increased resistance, the current through the fiber is greater in the first position than the second position as shown in FIG. 7 and generally decreases as a function of the distance of the fiber from the tip. Accordingly, current feedback may be used to assess a distance of the tapered fiber from the pipette tip in order to ensure proper fiber placement in an experimental setup. For example, the tapered fiber may be advanced until the current measurement is zero or near zero, thereby indicating proper placement of the tapered fiber at the tip.

### Example 4: Reconstruction Based on Photoacoustic and Fluorescence Feedback

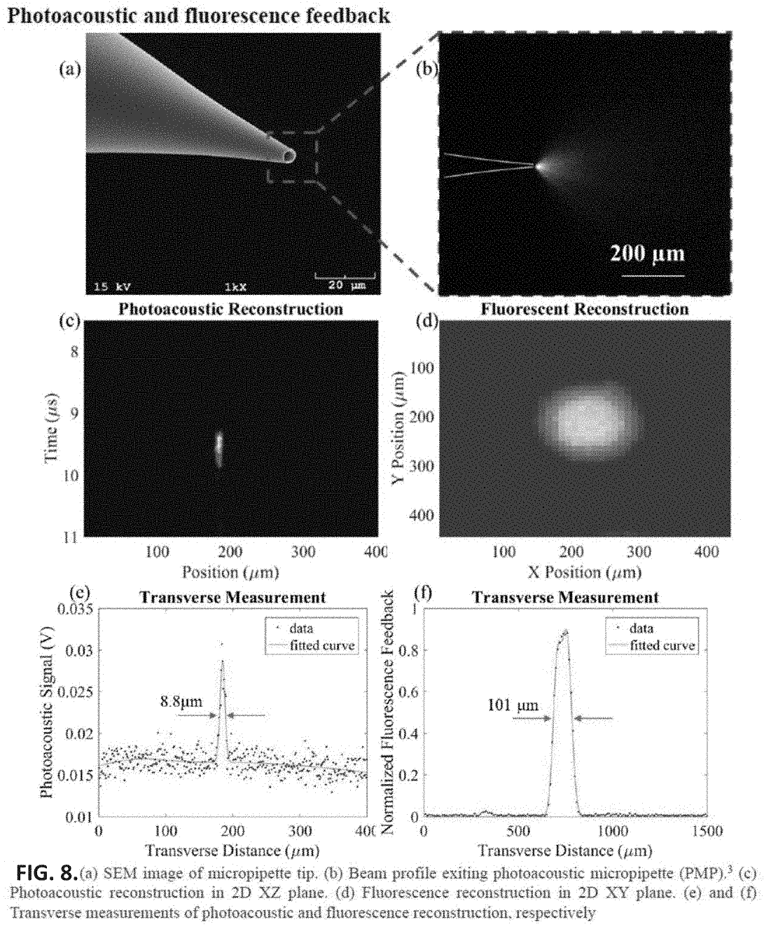

Methods: Fluorescence and photoacoustic feedback was collected in response to light emitted from a micropipette tip (FIG. 8 at (a) and (b)) and utilized to reconstruct an image and assess the position of a fluorescent bead.

Results: The photoacoustic and fluorescence feedback produced reconstructed images (FIG. 8 at (c) and (d)).

FIG. 8 shows a 2D image in the X,Z plane at (c). The 2D image was generated by raster scanning the micropipette tip along a 1D line over a 7.2 micron diameter carbon fiber thread. At each X position along the 1D line, a photoacoustic signal was recorded over a short time period. By measuring the time delay between the laser pulse and the photoacoustic signal reaching the transducer, the target position along the Z axis may be determined.

FIG. 8 shows a 2D image in the X,Y plane at (d). The micropipette tip was raster scanned along a 2D plane, where fluorescence signal was acquired at each position.

Transverse measurements of the reconstructions were fitted to curves (FIG. 8 at (e) and (f)).

### Example 5: Automated Approach Based on Fluorescence Feedback

Methods: Fluorescence feedback was collected by a probe in response to external illumination and utilized to reconstruct an image and assess the position of (1) a fluorescent bead and (2) a calcein AM-stained B35 neuroblastoma cell. A computer-automated system was utilized to localize to a fluorescent bead, and the neuroblastoma cell based on the fluorescence feedback. Customized LabVIEW software was used to raster scan the tip of the micropipette over a large area of interest (160×160 microns) at an initial distance above the sample. A software algorithm then causes the tip to descend and raster scans again at the new position, where the center of the scanning area corresponds to the peak fluorescent signal measured in the previous scan. The algorithm was repeated until the fluorescent signal reached a predetermined threshold. Thereafter, the scanning area was decreased to 25×25 microns and the algorithm was used to continue descent of the micropipette tip using the smaller scanning area until a second threshold was reached. Thereafter, the system was halted. For the fluorescent bead, the approach algorithm was performed three times from different starting positions defined by an XYZ coordinate system: (0,0,500), (0,500,500), and (0,−500,500). For the calcein AM-stained B35 neuroblastoma cell, the approach algorithm was performed from a starting position of 25 microns directly above the cell.

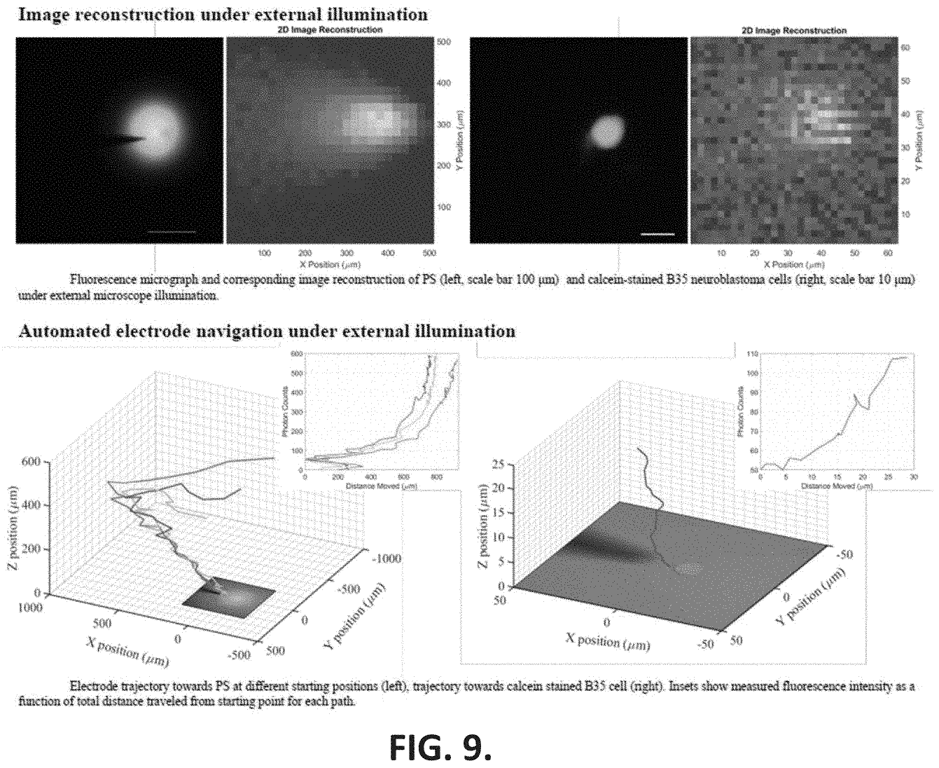

Results: Results are depicted in FIG. 9. For the fluorescent bead, results demonstrate that all three runs moved the micropipette towards the same angle of approach. The end point for all three runs were within 10 microns of each other. For the calcein AM-stained B35 neuroblastoma cell, the micropipette tip was successfully navigated toward the cell.

### Example 6: Automated Microscope-Independent Fluorescence Guided Micropipette

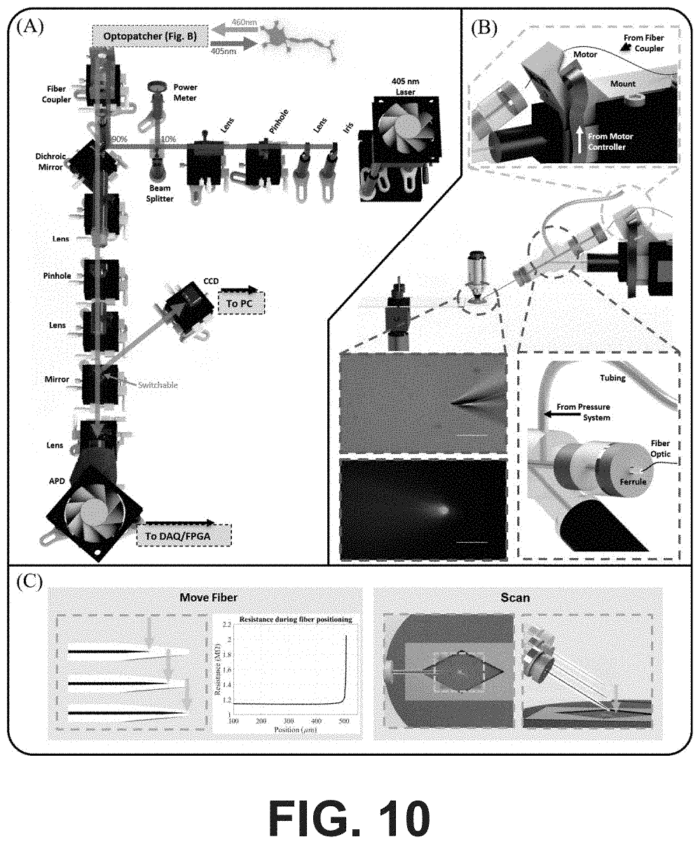

Methods: System Architecture Using Intra-Electrode Tapered Optical Fiber. The optical architecture is shown in FIG. 10 at (a). An ultra compact diode laser (IBEAM-SMART-405-S-HP; Toptica Photonics) operated at a 405 nm wavelength and 1-5 mW was used as the excitation source to generate fluorescence. The light source was spatially filtered using two lenses with a 50 mm focal length (ACN127-050-A and LA1131, Thorlabs) and a 50 μm pinhole (P50C, Thorlabs). To sample the power of the excitation beam over time, a beam splitter (BS037, Thorlabs) redirected 10% of the light towards a power meter (S120VC, Thorlabs). Light transmitted through the beam splitter enters an optical enclosure through an excitation filter (ET405/10x, Chroma). It was then redirected by a dichroic mirror (AT440DC, Chroma) to a fiber coupling system (F-91-C1-T, Newport), where the light was focused onto a tapered optical fiber (Nanonics) with a 1.5 μm diameter tip, 0.22 NA, and 125 μm core diameter utilizing a 10× objective (LMH-10x-532, Thorlabs). The tapered fiber passes through a light weight linear micro actuator (XLA-1-20-1250-T, Xeryon) and into an Optopatcher (A-M Systems) fitted with 270 μm inner diameter ferrule (CFLC270-10, Thorlabs). The tapered fiber was placed into a micropipette made from a borosilicate capillary tube with an outer diameter of 1.5 mm and an inner diameter of 1.1 mm. Micropipettes were pulled using a P-87 micropipette puller (Sutter Instruments) to have a 1-1.3 M resistance. Excitation light, having exited the tapered optical fiber, traveled through the micropipette tip onto the sample.

Emission light collected by the tapered fiber in the micropipette tip then traveled back into the optical enclosure where it was collimated by the fiber coupling system and passed through the dichroic mirror. The light was then focused with a 40× objective (89403-600, VWR), spatially filtered using a 25 μm pinhole (P25C, Thorlabs), and collimated by a lens with a 50 mm focal length (LA1131, Thorlabs). The light then passed through two emission filters (ET480/40m, Chroma; D460/50m, Chroma). A removable mirror (PF10-03-G01, Thorlabs) can be used to redirect the beam in the optical enclosure onto a CCD camera (DCC1645C, Thorlabs) for system alignment. When removed, the emitted light continued through a plano-convex lens (LA1131, Thorlabs) and was focused onto an avalanche photodiode (APD, SPCM-AQR, PerkinElmer). Data acquisition was performed with a multipurpose reconfigurable oscilloscope (NI PXIe-5170R, National Instruments Corporation).

Methods: Concentric Proximal Alignment of Tapered Optical Fiber with Micropipette Tip. Custom LabVIEW software was used to operate the system to achieve fluorescence guided automated neuronal approach. This robotic navigation was performed as a two-part system, including: i) tapered optical fiber positioning and ii) automated navigation of the micropipette as shown in FIG. 10 at (c). In order to assemble the probe, a micromanipulator (Patchstar, Scientifica) was used to carefully navigate the tapered optical fiber into the hollow core of an electrode. The micropipette was then manually fastened into the electrode holder. Micropipette resistance enables accurate positioning of the fiber tip at the electrode aperture. This was accomplished by submerging the micropipette tip inside a bath under application of low positive pressure (2-8 kPa) to avoid clogging. A voltage pulse was introduced into the micropipette and the resulting current was measured by a patch clamp amplifier (Model 2400, A-M Systems), thus providing a resistance measurement. The algorithm sends ASCII commands to a linear micro-actuator (XLA-1-20-1250-T, Xeryon) to move the tapered fiber over a range of 5 mm in 1.5 μm steps for optimal placement, as shown in FIG. 10 at (b) (50 μm scale bar shown for reference). When the micropipette resistance increases by roughly 1 MΩ, the algorithm stops moving the micro-actuator. This increase in resistance corresponds to accurate positioning of the tapered fiber for the micropipette architecture used. After each use, the micropipettes can be easily removed and replaced as described above.

Methods: Automated Fluorescence Guided Neuronal Approach. To monitor the automated navigation to the fluorescent target, these experiments were performed within the field of view of a DIC microscope (Eclipse FN1, Nikon). The micropipette tip was placed over the sample and the laser was left idle for 10 seconds, allowing the laser power and APD detection efficiency to stabilize. During experiments, the micropipette travels at a speed of ˜330 μm/s. Commands were sent to a micromanipulator (Patchstar, Scientifica) to scan a large region of interest (ROI) in 3 μm steps across the x-y plane, covering a 30×33 μm area. A photon count was performed at each step for 60 ms. Following each complete ROI scan there are two possible outcomes based on signal detection. Details of signal criteria are described further hereinbelow. If no signal was identified, the large ROI was centered around the x-y coordinate that corresponded to the peak photon count and the system descends 2 μm along the z-axis, where it performs another scan. This process continues until signal detection occurs. If signal was identified, the system switches to scanning a small ROI centered around the detected signal. This small ROI was scanned in 2 μm steps across the x-y plane, covering an 8×6 μm area. Micropipette resistance was measured at each step. After the initial small ROI scan, the micropipette descends 0.5 μm along the z-axis, where it performs another scan. This process continues until resistance increases by 0.1 MΩ, indicating physical contact between the micropipette tip and the target cell.

Methods: Image Processing Based Object Counting. In order to reliably switch scanning from a large ROI to a small ROI, one of two criteria must be met: i) photon count thresholding and ii) object counting. For the first criteria, the system assumes signal detection if the maximum photon count per scan increases by a user-defined value. The second criteria implements simple image processing techniques. Photon count rates are represented as an 8-bit image where the pixel location corresponds to each spatial coordinate. To generate a binary image of bright and dark pixels, a threshold was set at 75% between the maximum and minimum photon count rates. A flat linear structuring element, with an angle parallel to the micropipette in the x-y plane, was used to perform image dilation and subsequent image erosion. The resulting image was used to assess the presence of a signal through object counting. Neighboring bright pixels, either vertically, horizontally, or diagonally adjacent, are counted as a single object. If the object count was less than or equal to 4 the system assumes signal detection, thus triggering the switch to the small ROI. The threshold values were experimentally determined to provide accurate signal detection.

Methods: Cell Culture Preparation. The B35 cell line (rat neuroblastoma) was obtained from ATCC (CRL-2754, American Type Culture Collection). B35 cells were cultured in Dulbecco's Modified Eagle Medium (DMEM) supplemented with 10% FBS, 100 U/mL penicillin, and 100 μg/mL streptomycin and were maintained at 37° C. in a humidified 5% CO₂ incubator. For electrophysiology experiments, cells were seeded on glass coverslips in a 24 well plate at a density of 0.071×10⁶ and used within 72 hours. All cells used were at or below passage 5 in this study. Cells were stained with Hoechst 33342 (R37605, ThermoFisher Scientific) by incubating for ˜30 minutes. For the neuronal approach experiments, cells were submerged in artificial cerebrospinal fluid (aCSF) with the following components and concentrations: 135 mM NaCl, 2.5 mM KCl, 10 mM HEPES, 2 mM CaCl₂ and 1 mM MgCl₂. The intracellular pipette solution consisted of (in mM): 135 K gluconate, 4 KCl, 2 NaCl, 10 HEPES, 4 EGTA, and 0.3 Na Tris.

Results: Beam Profile and Optical Resolution of Intra-Electrode Tapered Optical Fiber. FIG. 10 shows the experimental setup for observing the beam profile (50 μm scale bar shown for reference). The beam shape of the light exiting the intra-electrode tapered optical fiber was imaged in ˜30 μM of fluorescein in DI water. Utilizing a FITC filter and an upright microscope, the emitted fluorescence from the 405 nm beam was captured. Concentric and proximal alignment of the tapered optical fiber and electrode tip was achieved using an optimized micropipette architecture. A resistance of 1.0-1.3 MΩ (see FIG. 10 at (c)) was obtained to facilitate proper placement, resulting in 1-4 μm distance between the fiber tip and electrode aperture.

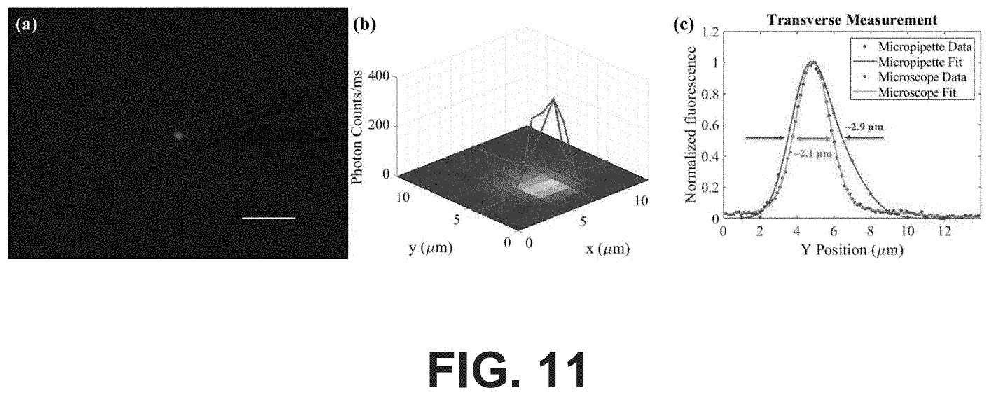

To better understand the resolution of the system, raster scans with 1 micron-sized steps were performed directly above fluorescent samples of known size. FIG. 11 at (a) depicts a microscope image of a single fluorescent bead (FMB-1.3 1-5 um, Cospheric), illuminated by excitation light exiting the tapered optical fiber (scale bar represents 25 μm). The raster scan was repeated while dropping 1 μm between each scan until the micropipette tip physically touched the fluorescent bead. The photon count acquired at each location along the scan was converted into an 8-bit pixel value as shown in the reconstruction in FIG. 11 at (b). The line plots correspond to the row and column of the reconstruction that contains the peak photon count. For simplicity, the z-axis begins at zero corresponding to the minimum signal. Full width half maximum (FWHM) measurements of the normalized data from the microscope and micropipette are shown FIG. 11 at (c). Using Matlab software to perform a Gaussian fit, the measured transverse FWHM is ˜2.1 μm for the microscope and ˜2.9 μm for the intra-electrode tapered optical fiber.

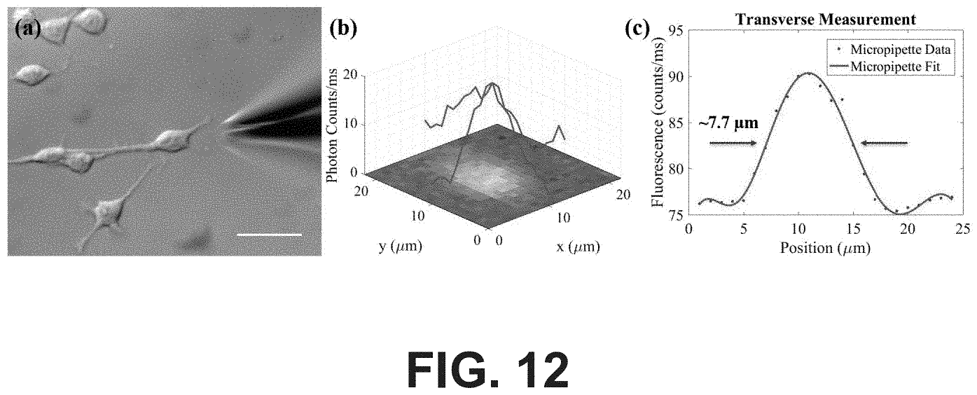

Further experiments were performed on cultured B35 neuroblastoma cells to test the resolution and sensitivity of the system. Shown in FIG. 12 at (a) is an overlayed image of brightfield (DIC Microscope) and fluorescence (scale bar represents 25 μm). Raster scans were performed with 1 micron-sized steps above the sample, collecting fluorescence from each position. The photon count acquired at each location along the large ROI scan is converted into an 8-bit pixel value as shown in the reconstruction in FIG. 12 at (b). The line plots correspond to the row and column of the reconstruction that contains the peak photon count. For simplicity, the z-axis begins at zero corresponding to the minimum signal. The FWHM of the cell was measured using both the microscope camera and the fluorescence guided system, as shown in FIG. 12 at (c). Using MATLAB software to perform a Gaussian fit, the measured transverse FWHM is ˜5.3 μm for the microscope and ˜7.7 μm for the intra-electrode tapered optical fiber.

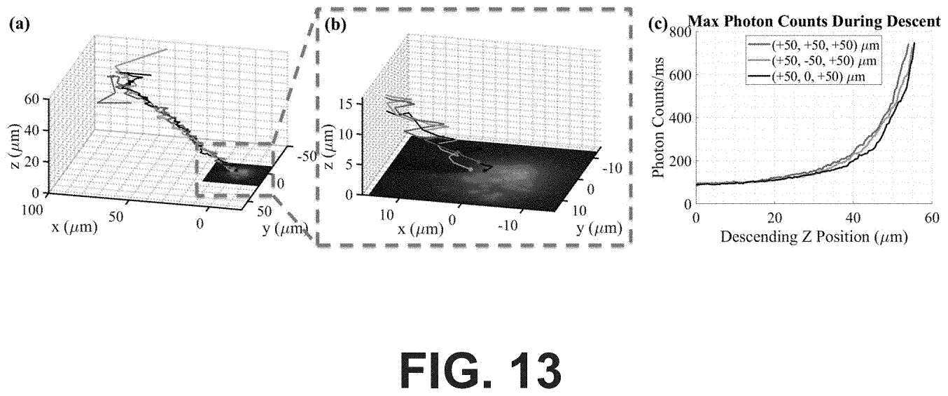

Results: Automated Navigational Approach. FIG. 13 depicts the results from an automated approach towards fluorescent beads. FIG. 13 demonstrates the trajectories toward the fluorescent beads. For clarity, only the position within each ROI scan corresponding to the peak fluorescent signal were plotted to produce the trajectories. Aggregated fluorescent beads with a cumulative diameter of ˜10 μm were utilized for this experiment. Since the beads cannot provide a reliable increase in resistance, a safe distance between the micropipette tip and the sample was maintained during automated navigation by using a photon count stop threshold. A photon count stop threshold of 700 photon counts/ms was experimentally determined by manually placing the tip near the fluorescent beads. From this position, the micropipette tip was moved +50 μm in the x, y, and z directions. The approach algorithm was run, until a stop threshold was reached (red trajectory). From this stop position, the micropipette tip was moved +50 μm in the x and z directions, however, was then shifted −50 μm in the y direction. The approach algorithm was again run from this starting position (green trajectory), until the stop threshold was reached. Lastly, the micropipette tip was moved +50 μm in the x and z directions, however, it was not moved in the y direction. Following this, the approach algorithm was run again (black trajectory). The starting points differed up to ˜100 μm, yet the algorithm placed the micropipette tips within 3 microns of each other, as shown in FIG. 13 at (b). As the micropipette tip approached the fluorescent beads the overall collected signal increases non-linearly, as shown in FIG. 13 at (c).

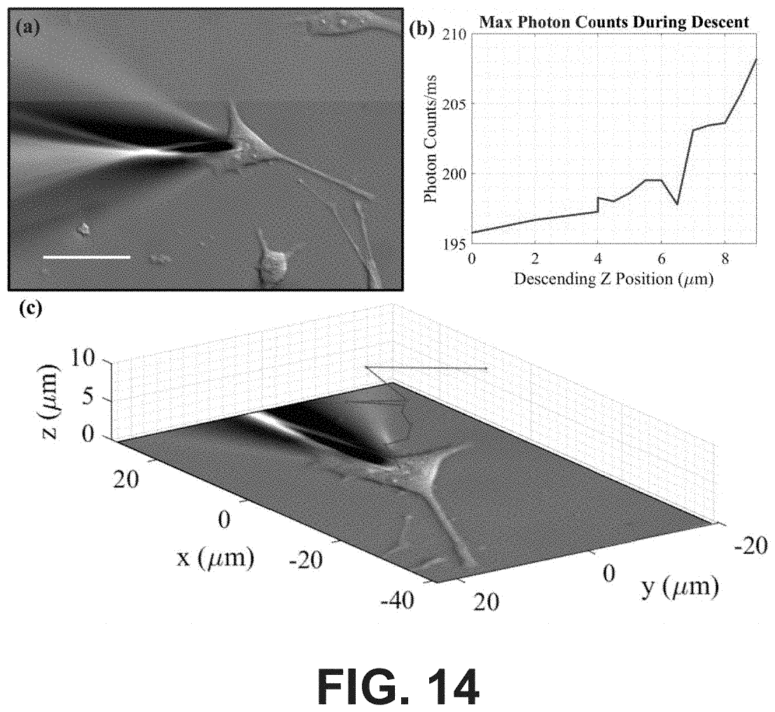

FIG. 14 depicts the results from automated neuronal approach towards fluorescently labeled cells. In contrast to the fluorescent beads an increase in resistance was utilized as the stop condition. The micropipette tip was coarsely aligned above a B35 neuroblastoma cell within the field of view of the DIC microscope and the algorithm was run. FIG. 14 at (a) is a representative DIC image of the final positioning of the micropipette. Similar to the fluorescent beads, the overall collected signal increased non-linearly as the labeled cells were approached, as shown in FIG. 14 at (b). FIG. 14 at (c) shows an overlayed brightfield (DIC) and fluorescent (DAPI) image and the coordinates that provide the peak signal per scan.

Discussion In this work, real-time automated navigation of a micropipette was performed using two-way fluorescence feedback. In order to accomplish this, excitation light was guided to the tip of the electrode through an integrated tapered optical fiber. As shown in FIG. 10 at (b), the beam profile takes a cone-like shape, where the fluorescent intensity is brightest at the tip and decreases with distance. This beam profile enables changes in fluorescent signal based on the proximity between the intra-electrode tapered optical fiber and the sample. To maximize the fluence of the beam on the sample, the fiber tip and electrode aperture are proximally aligned to have a distance between 1-4 μm. This distance enables high collection efficiency and allows a measurable increase in pipette resistance when the tip contacts the cell membrane. Resistances higher than 1.3 MΩ result in micropipette tapers that are too shallow for optimal fiber tip placement. This results in placement too far into the micropipette lumen, consequentially hindering fluorescence collection efficiency. Resistances lower than 1 MΩ allow the tip of the tapered fiber to go beyond the tip of the micropipette. This negatively affects resistance changes caused by close proximity between the electrode aperture and cell. In vivo patch clamping work is often performed with higher resistances (e.g. 5-8 MOhms). While not tested in this study, resistances beyond the upper and lower range used are likely possible by changing the micropipette or tapered optical fiber architecture.

In order to properly position the micropipette tip on a labeled cell, it is important to have sufficient spatial resolution and detection sensitivity. A ˜2.9 μm FWHM, as shown in FIG. 11 at (c), using the intra-electrode tapered optical fiber reveals a measurement that is smaller than the nucleus, and therefore the soma, of the B35 neuroblastoma cells. This indicates that the resolution of the system should be adequate for positioning the electrode aperture on a targeted neuron.

Sufficient detection sensitivity is demonstrated by scanning fluorescently labeled neurons, as shown in FIG. 12 at (a). However, substantial auto-fluorescence was generated in the tapered optical fiber by the 405 nm laser. Improved sensitivity may be achievable through the use of different laser lines and fluorophores.

An automated approach algorithm was developed to determine whether fluorescence intensity could be used as feedback to determine proximity between the micropipette tip and the sample. The algorithm starts by raster scanning a large ROI in the 2D x-y plane and subsequently descending along the z direction. After signal detection occurs, a small ROI scan is performed. At sufficiently close distances there is a relatively large increase in the fluorescence signal, as shown in FIG. 13 at (c). For these situations, a simple photon count threshold can determine signal detection. At larger distances between the intra-electrode tapered optical fiber and the sample, the increase in overall photon counts can be subtle. Here, signal detection can more readily be determined by including photon counts neighboring the peak signal, as opposed to a single spatial location. This is done by utilizing image processing-based object counting (see methods). A signal is determined by object counts below or equal to a threshold of 4. These results indicate that signal intensity threshold provides a sufficient method for approaching a target. Since large ROI scans took longer to complete than the small ROI scans, the overall approach time (<8 minutes for cells) was highly dependent on switching between scan sizes quickly. Prolonged approach times result in photobleaching of the cell, visible in real-time on the microscope camera as well as within the photon count rate. This method of determining signal detection resulted in successful automated approach to fluorescent beads and labeled neurons, as shown in FIGS. 13-14. These results indicate that the fluorescence intensity can be used as feedback to determine proximity between the micropipette tip and the sample, allowing for an automated approach.

Conclusion. We present a novel method for performing fluorescence guided automated neuronal approach capable of robotically positioning the micropipette electrode tip directly upon neurons in vitro. The system presented here is, to the best of our knowledge, the first to develop an intra-electrode tapered optical fiber for automated neuronal approach utilizing fluorescence feedback. The use of a tapered fiber to both provide excitation light and capture emitted fluorescence at the micropipette tip couples the feedback mechanism directly to the distance between the target and electrode aperture, while also allowing for the detection of cell-specific labels. This is performed independent of traditional microscope-based imaging approaches. The use of fluorescence is ubiquitous in neuroscience where fluorescent tags are often used to label specific neuronal subtypes or measure membrane potentials with ion or voltage indicators. Future work will investigate the use of intra-electrode tapered optical fibers in animal studies in vivo to target fluorescently labeled neurons for photometry, optogenetics, and patch clamping. Technologies that enable neuronal targeting beyond that of the working distance of microscope objectives will provide valuable insights into cellular activity in the deep brain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A system for navigating to a target neuron, the system comprising:
a recording electrode including:
a pipette having a hollow glass tip, and
a headstage configured to detect electrical resistance measurements at the hollow glass tip;
an actuator configured to move the hollow glass tip in one or more degrees of freedom;
at least one light source coupled to the recording electrode and configured to emit light from the hollow glass tip;
an ultrasound transducer configured to detect one or more photoacoustic signals in response to the light;
a light sensor configured to detect one or more optical signals in response to the light;
a processor; and
a non-transitory, computer-readable medium storing instructions that, when executed, cause the processor to:
perform one or more first iterations, each first iteration comprising:
receiving a first set of signals associated with the one or more photoacoustic signals from the ultrasound transducer,
receiving a second set of signals associated with the one or more optical signals from the light sensor,
calculating, based on at least one of the first set of signals and the second set of signals, a distance of the hollow glass tip with respect to the target neuron,
responsive to a determination that the distance is greater than a predetermined threshold, moving the hollow glass tip by a first increment in the one or more degrees of freedom via the actuator based on the distance, and
responsive to a determination that the distance is less than or equal to the predetermined threshold, maintaining the position of the hollow glass tip with respect to the target neuron via the actuator.

2. The system of claim 1, further comprising a suction source communicating with the hollow glass tip,
wherein the instructions, when executed, further cause the processor to activate the suction source, thereby forming a gigaohm seal between the hollow glass tip and the target neuron.

3. The system of claim 2, wherein the instructions, when executed, further cause the processor to control the suction source and the actuator to form one of a whole-cell patch clamp, a cell-attached patch clamp, an inside-out patch clamp, and an outside-out patch clamp between the recording electrode and the target neuron.

4. The system of claim 1, further comprising an optical fiber, wherein the at least one light source is coupled to the hollow glass tip by the optical fiber.

5. The system of claim 1, wherein the at least one light source comprises one or more of a pulsed laser and a modulated laser.

6. The system of claim 5, wherein the at least one light source comprises one or more of a neodymium-doped yttrium aluminum garnet laser and a titanium-sapphire laser.

7. The system of claim 1, wherein the at least one light source is configured to emit light at a plurality of wavelengths.

8. The system of claim 1, wherein the light sensor is an avalanche photodiode.

9. The system of claim 1, further comprising an amplifier configured to:
receive the one or more photoacoustic signals from the ultrasound transducer;
amplify the one or more photoacoustic signals to generate one or more amplified photoacoustic signals; and
communicate the amplified photoacoustic signals to the processor, wherein the first set of signals comprises the amplified photoacoustic signals.

10. The system of claim 1, wherein moving the hollow glass tip by a first increment in the one or more degrees of freedom comprises moving the hollow glass tip in the one or more degrees of freedom to increase one of an intensity of the one or more photoacoustic signals and an intensity of the one or more optical signals.

11. The system of claim 1, wherein the instructions, when executed, further cause the processor to perform one or more second iterations, each second iteration comprising:
receiving the electrical resistance measurements from the recording electrode;
calculating, based on the electrical resistance measurements, the distance of the hollow glass tip with respect to the target neuron, and
moving the hollow glass tip by a second increment in the one or more degrees of freedom via the actuator based on the distance.

12. The system of claim 11, wherein moving the hollow glass tip by a second increment in the one or more degrees of freedom comprises moving the hollow glass tip in the one or more degrees of freedom to increase a value of the electrical resistance measurements from the recording electrode.

13. The system of claim 1, wherein the ultrasound transducer is configured to detect the one or more photoacoustic signals across a range of at least 10 μm.

14. The system of claim 1, wherein the light sensor is configured to detect the one or more optical signals across a range of at least 10 μm.

15. The system of claim 1, wherein the target neuron is configured to emit the one or more optical signals based on genetic labeling of the target neuron.

16. A system for navigated to a target neuron, the system comprising:
a recording electrode including:
a pipette having a hollow glass tip, and
a headstage configured to detect electrical resistance measurements at the hollow glass tip;
an actuator configured to move the hollow glass tip in one or more degrees of freedom;
at least one light source coupled to the recording electrode and configured to emit light from the hollow glass tip;
an ultrasound transducer configured to detect one or more photoacoustic signals in response to the light;
a processor; and
a non-transitory, computer-readable medium storing instructions that, when executed, cause the processor to:
perform one or more first iterations, each first iteration comprising:
receiving a set of signals associated with the one or more photoacoustic signals from the ultrasound transducer,
calculating, based on the set of signals, a distance of the hollow glass tip with respect to the target neuron,
responsive to a determination that the distance is greater than a predetermined threshold, moving the hollow glass tip by a first increment in the one or more degrees of freedom via the actuator based on the distance, and
responsive to a determination that the distance is less than or equal to the predetermined threshold, maintaining the position of the hollow glass tip with respect to the target neuron via the actuator.

17. The system of claim 16, wherein the instructions, when executed, further cause the processor to perform one or more second iterations, each second iteration comprising:
receiving the electrical resistance measurements from the recording electrode;
calculating, based on the electrical resistance measurements, the distance of the hollow glass tip with respect to the target neuron, and
moving the hollow glass tip by a second increment in the one or more degrees of freedom via the actuator based on the distance.

18. A system for navigating to a target neuron, the system comprising:
a recording electrode including:
a pipette having a hollow glass tip, and
a headstage configured to detect electrical resistance measurements at the hollow glass tip;
an actuator configured to move the hollow glass tip in one or more degrees of freedom;
at least one light source coupled to the recording electrode and configured to emit light from the hollow glass tip;
a light sensor configured to detect one or more optical signals in response to the light;
a processor; and
a non-transitory, computer-readable medium storing instructions that, when executed, cause the processor to:
perform one or more first iterations, each first iteration comprising:
receiving a set of signals associated with the one or more optical signals from the light sensor,
calculating, based on the set of signals, a distance of the hollow glass tip with respect to the target neuron,
responsive to a determination that the distance is greater than a predetermined threshold, moving the hollow glass tip by a first increment in the one or more degrees of freedom via the actuator based on the distance, and
responsive to a determination that the distance is less than or equal to the predetermined threshold, maintaining the position of the hollow glass tip with respect to the target neuron via the actuator.

19. The system of claim 18, wherein the instructions, when executed, further cause the processor to perform one or more second iterations, each second iteration comprising:
receiving the electrical resistance measurements from the recording electrode;
calculating, based on the electrical resistance measurements, the distance of the hollow glass tip with respect to the target neuron, and
moving the hollow glass tip by a second increment in the one or more degrees of freedom via the actuator based on the distance.
